(19) United States
(12) Patent Application Publication
(10) Pub. No.: US 2008/0287514 A1
(43) Pub. Date: Nov. 20, 2008

(54) Topical compositions containing metronidazole

(76) Inventor: Jerry Zhang, Grayslake, IL (US)

Correspondence Address: JERRY ZHANG, 1061 CHADWICK DR., GRAYSLAKE, IL, 60030, US

(21) Appl. No.: 11/803,227

(22) Filed: May 14, 2007

(51) Int. Cl. A61K 31/4164

(52) U.S. Cl. 514/398

(57) ABSTRACT

Aqueous compositions containing solubilized metronidazole at a concentration of about 0.75% by weight or greater are able to be obtained by using the combination of solubility enhancing agents, one of which is niacinamide and the other is hydroxyalkyl urea. Related methods are also disclosed.

### CROSS-REFERENCE TO RELATED APPLICATIONS

Not Applicable

### FEDERALLY SPONSORED RESEARCH

Not Applicable

### SEQUENCE LISTING OR PROGRAM

Not Applicable

### FIELD OF INVENTION

This invention relates to topical compositions for treatment of dermal and mucosal disorders. In particular, the invention relates to topical compositions containing solubilized metronidazole as the active ingredient and the combination of niacinamide and hydroxyalkyl urea as the solubility enhancing agents, as well as related methods.

### BACKGROUND OF THE INVENTION

Metronidazole, chemical formula: C.sub.6H.sub.9 N.sub.3 O.sub.3 and molecular weight of 171.16 [chemical name: 1-(2-hydroxyethyl)-2-methyl-5-nitroimidazole], has long been known as an effective drug to treat a variety of disorders, and is especially known for the treatment of various protozoal diseases. As a topical therapy, metronidazole has also been shown to be useful in treating various skin disorders, including acne rosacea, bacterial ulcers, and perioral dermatitis. Metronidazole has been found to have anti-inflammatory activity when used topically to treat dermal disorders.

Compositions containing metronidazole as the active ingredient for treatment of dermal disorders are available in cream, lotion, and gel forms. For example, one commercially available metronidazole cream product, NORITATE™ (Dermik Laboratories, Inc., Berwyn, Pa.), contains 1% metronidazole in which the insoluble drug is suspended in the opaque cream. A commercially available metronidazole gel product, METROGEL™ (Galderma Laboratories, Ft. Worth, Tex.), offers two levels of metronidazole, 0.75% and 1%. The 0.75% gel product needs to be applied on at least twice daily. The 1% gel product contains solubilized metronidazole in presence of two solubility enhancing agents: beta-cyclodextrin and niacinamide. This product needs to be applied on only once daily. Metronidazole in the 1% gel is in the form of partial or complete inclusion complex with beta-cyclodextrin.

For the treatment of many dermal and mucosal disorders, it is often preferable to use compositions containing solubilized active ingredients, such as a solution, spray or gel, rather than a cream, lotion or an ointment. Creams, lotions, (typically oil-in-water emulsions) and ointments (typically petroleum jelly based compositions) are often comedogenic, acnegenic, or less cosmetically appealing to patients. Furthermore, active ingredient is generally more bioavailable in solubilized form than in insoluble, suspended or inclusion complex form.

U.S. Pat. Nos. 6,468,989 and 6,881,726 disclose using niacinamide and cyclodextrins as solubility enhancing agents to increase the solubility of metronidazole in water. Metronidazole forms partial or complete inclusion complex with the cyclodextrins. However, the complexed metronidazole needs to disassociate from the cyclodextrins to become free metronidazole.

Y. W. Chien, Journal of Parenteral Science and Technology, 38(1):32-36 (January 1984), discloses that water soluble vitamins, for example, niacinamide, ascorbic acid, or pyridoxine hydrochloride, are solubility enhancing agents that can increase solubility of metronidazole in water. However, these water soluble vitamins have disadvantages as the solubility enhancing agents. For example, ascorbic acid decomposes in water in presence of light and/or air. It does not address the combination of hydroxyalkyl urea and niacinamide as the solubility enhancing agents.

There is a need for solubility enhancing agents to increase the solubility of metronidazole in an aqueous composition without disadvantages of the prior art.

There is also a need for metronidazole composition with enhanced therapeutic efficacy and favorable cosmetic properties.

### SUMMARY OF THE INVENTION

It has been surprisingly discovered that the combination of niacinamide and hydroxyalkyl urea shows the synergistic effect in enhancing the aqueous solubility of metronidazole. The combination of niacinamide and hydroxyalkyl urea as the solubility enhancing agents offers greater solubility enhancing effect than either niacinamide or hydroxyalkyl urea as the solubility enhancing agent alone.

Accordingly, it is an object of the invention to provide a method for enhancing solubility of metronidazole in an aqueous composition to about 0.75% or greater by using the combination of niacinamide and hydroxyalkyl urea as the solubility enhancing agents.

Another object of the invention is to provide a method for preparing stable aqueous compositions containing solubilized metronidazole at a concentration of about 0.75% or greater. The method includes combining metronidazole and the combination of the solubility enhancing agents, one of which is niacinamide and the other is hydroxyalkyl urea, in an aqueous fluid.

Yet another object of the invention is to formulate stable aqueous compositions comprising solubilized metronidazole at a concentration of about 0.75% or greater and the combination of niacinamide and hydroxyalkyl urea as the solubility enhancing agents.

A further object of the invention is to formulate stable topical compositions for treating dermal and mucosal disorders, for example, rosacea, comprising a mixture of solubilized metronidazole at a concentration of about 0.75% or greater and the combination of niacinamide and hydroxyalkyl urea as the solubility enhancing agents.

Still other objects and advantages of the invention will, in part, be obvious and will, in part, be apparent from the following detailed description of the preferred embodiments.

### BRIEF DESCRIPTIONS OF THE DRAWINGS

FIG. 1 shows general structure of hydroxyalkyl urea.

FIG. 2 shows general structure of mono-substituted hydroxyalkyl urea.

### DETAILED DESCRIPTION OF THE PREFERRED EMBODIMENTS

It has been unexpectedly discovered that stable aqueous compositions containing metronidazole at a concentration of about 0.75% or greater are able to be obtained by using the combination of the solubility enhancing agents, wherein one of which is niacinamide and the other is hydroxyalkyl urea. The combination of the solubility enhancing agents in accordance with the present invention exhibits the synergistic effect in increasing the solubility of metronidazole in the aqueous compositions.

In the described embodiments, disclosed are methods for enhancing the solubility of metronidazole in the aqueous compositions to about 0.75% or greater by using the combination of the solubility enhancing agents, one of which is niacinamide and the other is hydroxyalkyl urea. Also disclosed are stable topical compositions containing solubilized metronidazole at a concentration of about 0.75% or greater obtained by using the combination of niacinamide and hydroxyalkyl urea as the solubility enhancing agents.

A ‘solubility enhancing agent’, as used herein, is a pharmaceutically acceptable chemical compound or a suitable combination of such compounds that when present in a solvent, increases the solubility of a second chemical compound, such as an active ingredient, in the solvent. The solubility enhancing agent is not itself a solvent for the second chemical compound.

The term ‘stable’, as used herein, means physical, rather than chemical stability. In accordance with the present invention, the metronidazole compositions are stable, that is substantially no crystallization or precipitation in the compositions, when stored at a refrigerated temperature for at least 7 days. The refrigerated temperature, in accordance with the present invention, is a temperature in the range of from just above the freezing temperature of the aqueous composition, which is about 0.degree. C., to about 10.degree. C.

The term ‘dissolved’, ‘dissolving’, ‘solubilized’ or ‘solubilizing’, when used in accordance with the present invention, means that an ingredient is substantially solubilized in the aqueous composition, and that the ingredient will not exist to any appreciable degree in the particulate, crystalline or droplet form in the composition.

As used herein, the term ‘about’ will be understood by persons of ordinary skill in the art and will vary to some extent on the context in which is used. If there are uses of the term which are not clear to persons of ordinary skill in the art given the context in which is used, ‘about’ will mean up to plus or minus 10% of the particular term.

The term ‘pharmaceutically acceptable’, as used herein, refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term ‘safe and effective’, as used herein, means a concentration of an active ingredient or an amount of a composition, that is sufficient enough to significantly and positively modify the condition to be treated but low enough to avoid serious side effects, within the scope of sound medical advice.

The term ‘metronidazole’, as used herein, is meant to include not only 1-(2-hydroxyethyl)-2-methyl-5-nitroimidazole, but also those analogs and derivatives of metronidazole (salts, esters, etc.) which are substantially solubilized in the compositions of the present invention and which have therapeutic activity when topically applied.

All percentages referred to in this specification are percentages by weight of the total composition unless otherwise indicated.

The metronidazole compositions of the present invention contain solubilized metronidazole at a concentration of at least 0.75%, at least 1.0%, at least 1.2%, or even as high as 2.5%, preferably at least 1.0%.

The solubility enhancing agents, niacinamide and hydroxyalkyl urea, are substantially solubilized in the aqueous composition of the present invention.

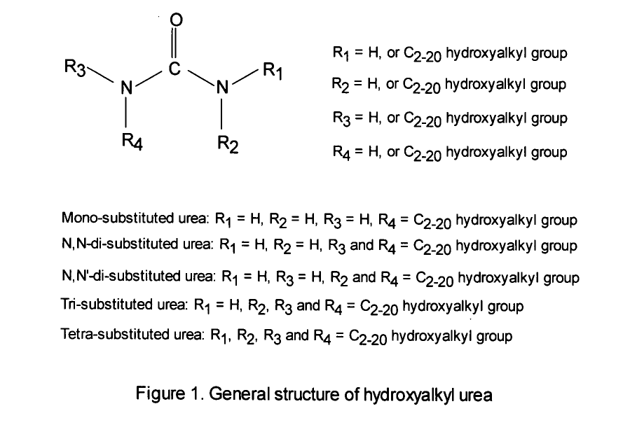

The general structure of hydroxyalkyl urea is shown in FIG. 1. All morphological forms of hydroxyalkyl urea, crystalline, semi-crystalline, and amorphous, are contemplated and within the scope of the present invention, either in admixture or in pure or substantially pure form. Furthermore, all stereoisomers of hydroxyalkyl urea are also contemplated and within the scope of the present invention. The definition of hydroxyalkyl urea in accordance with the present invention embraces all possible stereoisomers and their mixtures. It particularly embraces the racemic forms and the isolated optical isomers having the specified activity. The racemic forms are mixtures of the optically pure isomers. The individual optical isomers may be obtained from either asymmetric chemical synthesis or chiral separation methods such as, for example, chiral column chromatography, or co-crystallization with optically pure compounds.

Hydroxyalkyl urea may be mono-substituted hydroxyalkyl urea, di-substituted hydroxyalkyl urea, tri-substituted hydroxyalkyl urea, tetra-substituted hydroxyalkyl urea, or combinations thereof.

Examples of the suitable di-substituted hydroxyalkyl urea are N,N′-bis-(2-hydroxyethyl)urea, N,N-bis-(2-hydroxyethyl)urea, N,N′-bis-(3-hydroxypropyl)urea, N,N-bis-(2-hydroxypropyl)urea, N,N′-bis-(2-hydroxypropyl)urea, and combinations thereof.

Examples of the suitable tri-substituted hydroxyalkyl urea are N,N-bis-(2-hydroxypropyl)-N′-(2-hydroxyethyl)urea, N,N,N′-tris-(2-hydroxyethyl)urea, and combinations thereof.

Examples of the suitable tetra-substituted urea are N,N,N′,N′-tetrakis-(2-hydroxyethyl)urea, N,N,N′,N′-tetrakis-(2-hydroxypropyl)urea, and combinations thereof.

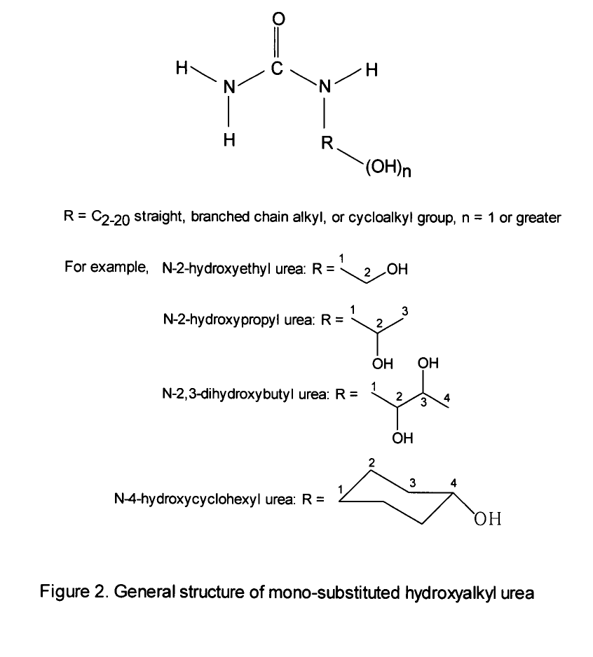

Among hydroxyalkyl urea, mono-substituted hydroxyalkyl urea is preferred. The mono-substituted hydroxyalkyl urea has the general structure as shown in FIG. 2.

The hydroxyalkyl group in hydroxyalkyl urea may be a straight, branched chain alkyl, or cycloalkyl group with one or more hydroxy groups attached onto the group at any suitable positions. Each hydroxyalkyl group may contain from 2 to 20 carbon atoms, preferably from 2 to 12 carbon atoms, more preferably from 2 to 8 carbon atoms. The hydroxyalkyl group contains at least one hydroxy group. The hydroxyalkyl group may have other suitable chemical group attached onto it at any suitable position. Examples of the suitable chemical group include ether, thioether, carboxy, amide, halogen, phenyl, or benzyl group, or suitable combinations thereof.

Examples of the suitable mono-substituted hydroxyalkyl urea are N-2-hydroxyethyl urea, N-3-hydroxypropyl urea, N-2-hydroxypropyl urea, N-2,3-dihydroxypropyl urea, N-4-hydroxybutyl urea, N-3-hydroxybutyl urea, N-2-hydroxybutyl urea, N-2,3-dihydroxybutyl urea, N-2,4-dihydroxybutyl urea, N-3,4-dihydroxybutyl urea, N-4-hydroxycyclohexyl urea, N-2,4-dihydroxycyclohexyl urea, and combinations thereof.

N-2-hydroxyethyl urea is most preferred mono-substituted hydroxyalkyl urea.

Niacinamide is a water soluble vitamin in vitamin B.sub.3 family. The combination of niacinamide and hydroxyalkyl urea exhibits the synergistic effect in increasing the solubility of metronidazole in the aqueous composition of the present invention.

The combination of hydroxyalkyl urea and niacinamide is used as the solubility enhancing agents in preparation of the aqueous composition of the present invention. The composition is substantially free of solubility enhancing agents other than hydroxyalkyl urea and niacinamide.

The overall concentration of the combination of niacinamide and hydroxyalkyl urea in the composition of the present invention is at least at a level sufficient to increase the solubility of metronidazole in the composition to a desired level, for example, to at least 0.75%, at least 1.0%, at least 1.2%, even as high as 2.5%. Any level of the overall concentration greater than the sufficient level is also suitable provided that concentration of each agent is below its water solubility limit and the stability, therapeutic efficacy, and cosmetic benefits of the composition are not negatively impacted. For example, the water solubility limit for niacinamide is about 50% by weight at 25.degree. C.

Hydroxyalkyl urea may be present in the composition of the present invention in an amount of at least 1%, at least 5%, at least 10%. The amount of hydroxyalkyl urea to be present is less than that which, without the presence of niacinamide, can enhance the solubility of metronidazole to the desired level.

In addition to its function as the solubility enhancing agent, hydroxyalkyl urea may also induce cosmetic benefits to the aqueous compositions. For example, N-2-hydroxyethyl urea is an excellent moisturizer.

The desired level of metronidazole in the composition is at least 0.75%, at least 1.0%, at least 1.2%, or even as high as 2.5%. Niacinamide may be present in the composition in an amount of at least 0.5%. The amount of niacinamide to be present is less than that which, without the presence of hydroxyalkyl urea, can enhance the solubility of metronidazole to the desired level.

The ratios of niacinamide to hydroxyalkyl urea in the composition may be any suitable ratios provided that the overall concentration of the solubility enhancing agents is at least at a level sufficient to solubilize metronidazole to the desired level and the concentration of each agent is less than that which, without presence of the other agent, can enhance the solubility of metronidazole to the desired level. The ratios of niacinamide to hydroxyalkyl urea in the composition may be varied depending on the desired level of solubilized metronidazole. Preferably, the concentration of hydroxyalkyl urea in the composition is equal to or greater than the concentration of niacinamide.

In a preferred embodiment, if a stable 1.0% metronidazole aqueous composition is desired, a combination of 1% to 2% niacinamide and 5% to 10% hydroxyalkyl urea may be used to induce the solubility enhancing effect.

In a preferred method of preparing aqueous composition of the present invention, a stable aqueous solution of niacinamide and hydroxyalkyl urea is prepared, wherein the concentrations of niacinamide and hydroxyalkyl urea are sufficient to solubilize metronidazole to the desired level. Metronidazole is combined with the solution containing the dissolved solubility enhancing agents. The mixture is stirred or agitated at room temperature until metronidazole is dissolved. An elevated temperature, preferably a temperature in the range of from about 40.degree. C. to about 80.degree. C., may be used to facilitate the solubilization of metronidazole and then after the solubilization, the solution is permitted to cool to room temperature.

The aqueous composition, in accordance with the present invention, may be in the form of a solution, spray or gel. Preferably the composition is a gel. Therefore, the aqueous composition preferably contains a gelling agent. Any gelling agent that is dispersible in the aqueous vehicle, physically and chemically compatible with the ingredients in the composition, and forms an aqueous gel of substantially uniform consistency is suitable for use in the present invention.

In one embodiment, the gel form of the present invention may be prepared by dispersing the suitable gelling agent in the aqueous solution containing the dissolved metronidazole and solubility enhancing agents to form the gel. In another embodiment, the gel form of the present invention may be prepared by dispersing the aqueous solution containing the dissolved metronidazole and solubility enhancing agents in a pre-made aqueous gel.

Examples of the suitable gelling agents are polycarbohydrate based gelling agents and polyacrylic acid based gelling agents. Examples of the suitable polycarbohydrate gelling agents are hydroxyethylcellulose, hydroxypropylcellulose, and xanthan gum. Examples of the suitable polyacrylic acid gelling agents are CARBOPOL Brand 934, 940, 941, Ultrez 10, and Ultrez 20 (available from Noveon Corp., Cleveland, Ohio). Combinations of the polycarbohydrate gelling agents and/or polyacrylic acid gelling agent are also suitable as the gelling agents.

The pH of the composition of the present invention is preferably kept at or below 7.0. The pH of metronidazole in water is in the range of about 5.0 to about 6.0. However, other ingredients present in the aqueous compositions may affect the pH of the composition. If pH adjustment is necessary, any pharmaceutically acceptable inorganic or organic acid or base may be used to adjust pH of the composition. The pH of the aqueous composition may be kept in the range of from about 4.0 to about 7.0, preferably from about 4.5 to about 6.5, more preferably from about 5.0 to about 6.0.

The pH of the composition may be maintained by using a suitable buffer system provided that the buffer system is physically and chemically compatible with the ingredients in the composition, does not substantially decrease the solubility of metronidazole in the composition, or reduces the therapeutic efficacy of the composition.

The suitable buffer system includes any pharmaceutically acceptable buffer system capable of maintaining the pH of the composition in the range of about 4.0 to about 7.0 with sufficient buffering capacity. Examples of the suitable buffer system include, but not limited to: lactate buffer with useful pH range of about 4.0 to 5.5, citrate buffer with useful pH range of about 4.0 to about 6.0, or citrate phosphate buffer with useful pH range of about 4.0 to about 7.0.

The composition may contain other pH stabilization agents. Examples of the pH stabilization agents include, but not limited to: low molecular weight esters and lactones.

Examples of the low molecular weight esters are ethyl or propyl esters of organic acids, such as triethyl citrate and ethyl lactate. Examples of the lactones are pantolactone and D-gluconolactone. The pH stabilization agents may be present in an amount of from about 0.1% to about 5%, preferably from about 0.2% to about 2%.

The aqueous composition, in accordance with the present invention, may contain conventional amounts of customary auxiliaries and additives provided that such auxiliaries and additives are physically and chemically compatible with the ingredients in the composition, do not substantially decrease the solubility of metronidazole in the composition, or reduce the therapeutic efficacy of the composition. The term, ‘substantially decrease’, as used herein, means that inclusion of the auxiliaries and additives decreases the solubility of metronidazole to less than about 0.75% in the aqueous compositions. Although it is possible to offset the decrease of the solubility of metronidazole by increasing overall concentration of the solubility enhancing agents, it is preferable that the auxiliaries and additives do not substantially decrease the solubility of metronidazole in the composition.

Furthermore, the auxiliaries and additives do not substantially impair favorable cosmetic properties of the aqueous composition of the present invention. The total concentration of all auxiliaries and additives in the aqueous composition may be up to about 20%, preferably up to about 15%, more preferably up to about 10%.

Examples of the customary auxiliaries and additives include, but not limited to: moisturizing compounds, film-forming polymers, and other desirable ingredients. The customary auxiliaries and additives are substantially solubilized in the composition of the present invention.

The aqueous composition of the present invention may contain moisturizing compounds including, but not limited to: allantoin, polyhydric alcohols (also known as polyols), polyol ethers and esters, low molecular weight polyethylene glycols, lactates, sugars, methyl glucose ethers, sodium pyrrolidone carboxylic acid, sodium hyaluronate, panthenol, hyaluronic acid, alpha.- and beta.-hydroxy acids, or combinations of the suitable moisturizing compounds.

Allantoin, known for its therapeutic action on skin, has been widely used for decades in cosmetic and pharmaceutical formulations. Important features of allantoin are keratolytic action and promotion and acceleration of cell proliferation. Allantoin is also used for its soothing and anti-irritating properties. Allantoin may be present in an amount of from about 0.1% to about 5%, preferably from about 0.2% to about 2%.

Examples of the suitable polyols are glycerin (also known as glycerol), propylene glycol (also known as 1,2-propanediol), 1,3-propanediol, 1,2-butanediol, 1,3-butanediol, 1,4-butanediol, 2,3-butanediol, 1,2-pentanediol, 1,5-pentanediol, 1,2-hexanediol, 2-methyl-2,4,-pentanediol (also known as hexylene glycol), 1,2-hexanediol, 1,6-hexanediol, diethylene glycol, diglycerin, dipropylene glycol, triethylene glycol, 1,2,3-hexanetriol, 1,2,6-hexanetriol, or combinations of the suitable polyols in any given ratio. Preferred polyols are glycerin, propylene glycol, 1,4-butanediol, and hexylene glycol. Examples of the suitable low molecular weight polyethylene glycols (PEG) are PEG-4, PEG-6, PEG-8, and PEG-10. Examples of the suitable lactates are ammonium lactate, sodium lactate, and potassium lactate. Examples of the suitable methyl glucose ethers are methyl gluceth-10 and methyl gluceth-20. Examples of the suitable alpha.- and beta.-hydroxy acids are glycolic acid, lacetic acid, mandelic acid, and salicylic acid. The moisturizing compounds mentioned here may be present in an amount up to about 20%, preferably up to about 15%, more preferably up to about 10%.

The film forming properties of polymers help maintain active ingredients on the site of application, which may help enhance therapeutic efficacy of the composition. Examples of the suitable film forming polymers are hydroxypropyl cellulose, polyvinylpyrrolidone, polyvinylpyrrolidone vinylacetate copolymer, polyvinyl alcohol, and combinations thereof.

The film forming polymers may be present in an amount of from about 0.1% to about 10%, preferably from about 0.5% to about 5%.

The aqueous composition of the present invention may also contain one or more other desirable ingredients including, but not limited to: skin penetration enhancers, herbal extracts, chelating agents, preservatives, colorants, fragrances, and so on. Examples of the suitable herbal extracts are grape seed extract and onion extract. Examples of the suitable skin penetration enhancers are ethanol, isopropanol, propylene glycol, dimethyl isosorbide, and N-methyl-1-pyrrolidone. Examples of the suitable chelating agents are EDTA (ethylenediaminetetraacetic acid), EGTA [ethylenebis(oxyethylenenitrilo)tetraacetic acid], and pharmaceutically acceptable salts thereof.

The aqueous composition of the present invention may be used for topical treatment of dermal or mucosal disorders that are responsive to therapy with metronidazole. In accordance with the method of treatment of the present invention, a stable aqueous composition containing metronidazole at a concentration of about 0.75% or greater, preferably about 1.0% or greater, is topically applied in a safe and effective amount to skin or mucosal surfaces of human beings or animals in need of such therapy.

The therapeutic method of the present invention may be used to treat any disorder that is responsive, or potentially responsive, to metronidazole therapy. Examples of the disorders that are suitable to be treated include inflammatory lesions on the skin, oral mucosa, or vaginal mucosa, and certain infectious diseases that may be treated topically.

Preferably, the disorder to be treated is rosacea.

The following examples are included for purposes of illustrating the technology covered by this disclosure. They are not intended to limit the scope of the claimed invention in any manner. One skilled in the art will understand that there are alternatives to these specific embodiments that are not completely described by these examples.

### EXAMPLE 1

The solubility enhancing effect of the combination of niacinamide and hydroxyalkyl urea in increasing the solubility of metronidazole in the aqueous compositions is evaluated by physical stability of the prepared compositions.

Therefore, the compositions in accordance with the present invention are subject to a physical stability test. The protocol for the stability test is as follows: metronidazole and the solubility enhancing agents are combined with water either at room temperature or elevated temperature while stirring until dissolved. The solutions are permitted to cool to room temperature. Customary auxiliaries and additives may be added to the aqueous solutions containing solubilized metronidazole and solubility enhancing agents. The solutions may be gelled by using the suitable gelling agents. The compositions both in gel or solution form are then packaged in clear glass vials and sealed. The vials are kept at room temperature for 24 hours and monitored for crystal formation or precipitation. After 24 hours, the vials are placed in a refrigerator maintained at a temperature of about 4.degree. C. for 7 days. After 7 days, the vials are removed from the refrigerator. Evidence of crystal formation or precipitation in the compositions is examined. The test results are recorded: ‘clear’ means no crystals or precipitates; ‘crystals formed’ or ‘precipitates formed’ means that crystals or precipitates are formed in the compositions.

### EXAMPLE 2 (FOR COMPARISON)

This example is to establish the baseline data for niacinamide as the only solubility enhancing agent.

| Niacinamide | Metronidazole | Results from stability test |
|---|---|---|
| 2% | 1.0% | Crystals formed |
| 3% | 1.0% | Crystals formed |
| 4% | 1.0% | Crystals formed |
| 5% | 1.0% | Clear |

Each formulation was prepared by combining niacinamide and metronidazole in water (q.s 100%). The mixture was kept at a temperature of about 45.degree. C. while stirring until dissolved. The solutions were permitted to cool to room temperature, packaged and sealed in glass vials. The stability tests described in Example 1 were performed and the test results were recorded.

This example shows that the concentration of niacinamide required to obtain a stable aqueous solution of 1.0% metronidazole is about 5%.

### EXAMPLE 3 (FOR COMPARISON)

This example is to establish the baseline data for hydroxyalkyl urea as the only solubility enhancing agent. N-2-hydroxyethyl urea was used as the example.

| N-2-hydroxyethyl urea | Metronidazole | Results from stability test |
|---|---|---|
| 13% | 1.0% | Crystals formed |
| 14% | 1.0% | Crystals formed |
| 15% | 1.0% | Crystals formed |
| 16% | 1.0% | Clear |

Each formulation was prepared by combining N-2-hydroxyethyl urea and metronidazole in water (q.s. 100%). The mixture was kept at a temperature of about 45.degree. C. while stirring until dissolved. The solutions were permitted to cool to room temperature, packaged and sealed in glass vials. The stability tests described in Example 1 were performed and the test results were recorded.

This example shows that the concentration of N-2-hydroxyethyl urea required to obtain a stable aqueous solution of 1.0% metronidazole is about 16%.

### EXAMPLE 4

This example is to demonstrate the synergistic solubility enhancing effect in increasing the solubility of metronidazole by using the combination of niacinamide and hydroxyalkyl urea at various concentrations. N-2-hydroxyethyl urea was used as the example.

| | | | Results from |
|---|---|---|---|
| N-2-hydroxyethyl urea | Niacinamide | Metronidazole | stability test |
| 1.0% | 3.0% | 1.0% | Crystals |
| 1.0% | 4.0% | 1.0% | Clear |
| 4.0% | 1.0% | 1.0% | Crystals |
| 5.0% | 1.0% | 1.0% | Clear |
| 5.0% | 2.0% | 1.0% | Clear |
| 3.0% | 3.0% | 1.0% | Clear |
| 10.0% | 2.0% | 1.2% | Clear |

Each formulation was prepared by combining N-2-hydroxyethyl urea, niacinamide, and metronidazole in water (q.s. 100%). N-2-hydroxyethyl urea is available from a number of commercial sources, for example, Aldrich Chemical Company (Milwaukee, Wis.). The mixture was kept either at room temperature or at a temperature of about 45.degree. C. while stirring until dissolved. The solutions were permitted to cool to room temperature, packaged and sealed in glass vials. The stability tests described in Example 1 were performed and the test results were recorded.

The results from Examples 2 and 3 show that about 5% niacinamide or about 16% N-2-hydroxyethyl urea is needed to obtain a stable 1.0% aqueous solution of solubilized metronidazole, respectively. This suggests that 1% niacinamide is functionally equivalent to about 3.2% N-2-hydroxyethyl urea in inducing the solubility enhancing effect. Therefore, for the composition containing 5.0% N-2-hydroxyethyl urea, 1.0% niacinamide, and 1.0% metronidazole, if there were no synergistic effect, it would have been functionally equivalent to a composition containing 5.0% N-2-hydroxyethyl urea, 3.2% N-2-hydroxyethyl urea, and 1.0% metronidazole. The overall concentration of 8.2% N-2-hydroxyethyl urea is not sufficient to solubilize 1.0% metronidazole as demonstrated in Example 3. However, the result from the stability test in this example demonstrates that the composition containing 5.0% N-2-hydroxyethyl urea, 1.0% niacinamide, and 1.0% metronidazole is indeed stable. The result clearly suggests that the synergistic solubility enhancing effect exists when the combination of niacinamide and hydroxyalkyl urea is used as the solubility enhancing agents in increasing the solubility of metronidazole in the aqueous compositions. The synergistic solubility enhancing effect is also observed in rest of the stable compositions shown in this example.

The results also show that if concentration of N-2-hydroxyethyl urea is 1.0%, a concentration of about 4.0% of niacinamide is needed to obtain a stable aqueous solution of 1.0% metronidazole. If concentration of niacinamide is 1.0%, a concentration of about 5.0% or more of N-2-hydroxyethyl urea is needed to obtain a stable aqueous solution of 1.0% metronidazole.

### EXAMPLE 5

This example is to formulate a stable 1.0% metronidazole gel suitable for treatment of dermal and mucosal disorders in accordance with the present invention.

| Component | Amount |
|---|---|
| N-2-hydroxyethyl urea | 6.0% |
| Niacinamide | 2.0% |
| Metronidazole | 1.0% |
| Germaben - II | 0.5% |
| Propylene glycol | 2.0% |
| Hydroxyethyl cellulose | 1.25% |
| Water | 87.25% |

N-2-hydroxyethyl urea, niacinamide, and metronidazole were added to water. The mixture was kept at about 45.degree. C. while stirring until dissolved. The solution was permitted to cool to room temperature. Propylene glycol and Germaben-II were added and solubilized. Germaben-II is an antimicrobial preservative system consisting of propylene glycol and diazolidinyl urea and methylparaben and propylparaben (available from ISP Corp. Wayne, N.J.). Add water to q.s. the batch to final weight. Hydroxyethyl cellulose (Natrosol CS 250HR, Hercules Inc. Wilmington, Del.) was shifted into the solution. The mixture was kept at room temperature while stirring until gelled. A clear gel was formed. The pH of the gel composition was about 6.0. The sample underwent the stability test. The test result showed that no evidence of crystallization or precipitation was evident, indicating that the gel was stable. The gel is suitable for use to treat rosacea.

### EXAMPLE 6

This example is to formulate a stable 1.0% metronidazole gel suitable for treatment of dermal and mucosal disorders in accordance with the present invention.

| Component | Amount |
|---|---|
| N-2-hydroxyethyl urea | 6.0% |
| Niacinamide | 1.0% |
| Metronidazole | 1.0% |
| EDTA disodium | 0.05% |
| Germaben - II | 0.5% |
| Glycerin | 5.0% |
| Hydroxyethyl cellulose | 1.25% |
| Water | 85.2% |

N-2-hydroxyethyl urea, niacinamide, EDTA disodium, and metronidazole were added to water. The mixture was kept at a temperature of about 45.degree. C. while stirring until dissolved. The solution was permitted to cool to room temperature. Glycerin and Germaben-II were added and solubilized. Add water to q.s. the batch to final weight. Hydroxyethyl cellulose was shifted into the solution. The mixture was kept at room temperature while stirring until gelled. A clear gel was formed. The pH of the gel composition was about 6.0. The sample underwent the stability test. The test result showed that no evidence of crystallization or precipitation was evident, indicating that the gel was stable. The gel is suitable for use to treat rosacea.

It will thus be seen that the objects set forth above, among those made apparent from the preceding description, are efficiently attained and, since certain changes may be made in carrying out the above process and in the composition set forth without departing from the spirit and scope of the invention, it is intended that all matter contained in the above description shall be interpreted as illustrative and not in a limiting sense.

It is also to be understood that the following claims are intended to cover all of the generic and specific features of the invention herein described and all statements of the scope of the invention which, as a matter of language, might be said to fall there between.

Particularly it is to be understood that in the claims, ingredients or compounds recited in the singular are intended to include compatible mixtures of such ingredients wherever the sense permits.

What is claimed is:

1. A composition, comprising:
metronidazole;
niacinamide;
hydroxyalkyl urea;
an aqueous vehicle.

2. The composition of claim 1 wherein the hydroxyalkyl urea is mono-substituted hydroxyalkyl urea.

3. The composition of claim 1 wherein the hydroxyalkyl urea is N-2-hydroxyethyl urea.

4. The composition of claim 1 wherein niacinamide is in an amount of about 0.5% by weight or greater.

5. The composition of claim 1 wherein the hydroxyalkyl urea is in an amount of about 1.0% by weight or greater.

6. The composition of claim 1 wherein metronidazole is in an amount of about 0.75% by weight or greater.

7. The composition of claim 1 wherein metronidazole is in an amount of about 1.0% by weight or greater.

8. The composition of claim 1 further comprising a gelling agent.

9. An aqueous composition for therapeutic treatment of dermal and mucosal disorder including administering to a patient in need of such treatment a safe and effective amount of the composition, comprising metronidazole, niacinamide, and hydroxyalkyl urea, wherein the combination of niacinamide and the hydroxyalkyl urea is in an amount sufficient to solubilize metronidazole in the composition.

10. The composition of claim 9 wherein the hydroxyalkyl urea is mono-substituted hydroxyalkyl urea.

11. The composition of claim 9 wherein the hydroxyalkyl urea is N-2-hydroxyethyl urea.

12. The composition of claim 9 wherein niacinamide is in an amount of about 0.5% by weight or greater.

13. The composition of claim 9 wherein the hydroxyalkyl urea is in an amount of about 1.0% by weight or greater.

14. The composition of claim 9 wherein the concentration of metronidazole is about 0.75% by weight or greater.

15. The composition of claim 9 wherein the concentration of metronidazole is about 1.0% by weight or greater.

16. The composition of claim 9 wherein the disorder is rosacea.

17. The composition of claim 9 further comprising a gelling agent.

18. A method for increasing the solubility of metronidazole in an aqueous vehicle, comprising combining metronidazole, niacinamide, and hydroxyalkyl urea in an aqueous fluid, wherein the combination of niacinamide and hydroxyalkyl urea is in an amount sufficient to solubilize metronidazole.

19. The method of claim 18 wherein the solubility of metronidazole is increased to about 0.75% by weight or greater.

20. The method of claim 18 wherein the solubility of metronidazole is increased to about 1.0% by weight or greater.

21. The method of claim 18 wherein niacinamide is in an amount of about 0.5% by weight or greater.

22. The method of claim 18 wherein the hydroxyalkyl urea is in an amount of about 1.0% by weight or greater.

23. The method of claim 18 wherein the hydroxyalkyl urea is mono-substituted hydroxyalkyl urea.

24. The method of claim 18 wherein the hydroxyalkyl urea is N-2-hydroxyethyl urea.
